(19) United States
(12) Patent Application Publication
(10) Pub. No.: US 2014/0126685 A1
(43) Pub. Date: May 8, 2014

(54) ANGIOGRAPHIC EXAMINATION METHOD

(71) Applicants: Yu Deuerling-Zheng, Forchheim (DE); Michael Manhart, Furth (DE)

(72) Inventors: Yu Deuerling-Zheng, Forchheim (DE); Michael Manhart, Furth (DE)

(21) Appl. No.: 14/014,608

(22) Filed: Aug. 30, 2013

Related U.S. Application Data
(60) Provisional application No. 61/722,916, filed on Nov. 6, 2012.

(30) Foreign Application Priority Data
Nov. 2, 2012 (DE) 102012220028.2

(51) Int. Cl. A61B 5/00

(52) U.S. Cl. CPC A61B 5/0033
USPC 378/4

(57) ABSTRACT

An angiographic examination method for examining an organ, vascular system or other regions of a patient is proposed. Projection images are acquired by an angiography system having an X-ray tube assembly and an X-ray image detector applied to ends of a C-arm, a patient table having a tabletop for carrying the patient, a system control unit, an imaging system and a monitor. Projection images are generated by rotational angiography from a plurality of projection angles. The projection images are subjected to a pre-processing of an FDK reconstruction, the result of which is filtered by a noise-reduction method. A predetermined number of dynamic, iterative reconstruction steps is carried out. Time attenuation curves are reconstructed, which are modeled with a weighted sum of linear basis functions.

### CROSS REFERENCE TO RELATED APPLICATIONS

This application claims benefit of provisional patent application 61/722,916 filed on Nov. 6, 2012, the entire content of which is hereby incorporated by reference. This application also claims priority to German application No. 10 2012 220 028.2 DE filed Nov. 2, 2012, the entire content of which is hereby incorporated herein by reference.

### FIELD OF INVENTION

The invention relates to an angiographic examination method of an organ, vascular system or other regions of the body as an examination object of a patient by means of an angiography system having an X-ray emitter, an X-ray image detector, which are applied to the ends of a C-arm, a patient table having a tabletop for carrying the patient, a system control unit, an imaging system and a monitor, wherein projection images are generated by means of rotational angiography from a plurality of projection angles.

### BACKGROUND OF INVENTION

An angiography system for carrying out such an angiographic examination method is, for example, known from U.S. Pat. No. 7,500,784 B2, which is illustrated below in FIG. 1.

FIG. 1 shows a monoplane X-ray system, depicted as an example, having a C-arm 2 held by a stand 1 in the form of an industrial or jointed-arm robot with six axes, on the ends of which C-arm an X-radiation source, for example an X-ray emitter 3 having an X-ray tubes and collimator, and an X-ray image detector 4, are applied as an image recording unit.

By means of the jointed-arm robot known, for example, from U.S. Pat. No. 7,500,784 B2, which preferably has six axes of rotation and thus six degrees of freedom, the C-arm 2 can be spatially adjusted in any way, for example by rotating it around a centre of rotation between the X-ray emitter 3 and the X-ray image detector 4. The angiographic X-ray system 1 to 4 according to the invention can, in particular, be rotated around centers of rotation and axes of rotation in the C-arm plane of the X-ray image detector 4, preferably around the centre point of the X-ray image detector 4 and around the centre point of the axes of rotation that cut the X-ray image detector 4.

The known jointed-arm robot has a base frame that is, for example, mounted fixedly to a base. A rotating table is fastened to this for rotation around a first axis of rotation. A robotic swing arm is applied to the rotating table to swivel around a second axis of rotation, to which a robotic arm is fastened for rotation around a third axis of rotation. A robotic hand is applied to the end of the robotic arm for rotation around a fourth axis of rotation. The robotic hand has a fastening element for the C-arm 2, which can be swiveled around a fifth axis of rotation and rotated around a sixth axis of rotation that runs perpendicular thereto.

The implementation of the X-ray diagnostic apparatus is not dependent on the industrial robot. Typical C-arm devices can also be used.

The X-ray image detector 4 can be a rectangular or square, flat semiconductor detector, which is preferably produced from amorphous silicon (a-Si). However, integrating and potentially counting CMOS detectors can also be applied.

A patient 6 to be examined is located as the examination object on a tabletop 5 of a patient table in the beam path of the X-ray emitter 3. A system control unit 7 having an imaging system 8 is connected to the X-ray diagnostic apparatus, which receives and processes the image signals of the X-ray image detector 4 (operating elements are, for example, not depicted). The X-ray images can then be observed on displays of a monitor light 9. Furthermore, a calculation device 10 is provided in the system control unit 7, the function of which will be described in greater detail.

Instead of the X-ray system depicted, for example, in FIG. 1, having the stand 1 in the form of the industrial or jointed-arm robot with six axes, as is depicted in simplified form in FIG. 2, the angiographic X-ray system can also have a normal ceiling-mounted or base-mounted holder for the C-arm 2.

Instead of the C-arm 2 depicted as an example, the angiographic X-ray system can also have separate ceiling-mounted and/or base-mounted holders for the X-ray emitter 3 and the X-ray image detector 4, which, for example, are rigidly coupled electronically.

The X-ray emitter 3 emits a beam 11 departing from a beam focus of its X-radiation source, which beam strikes the X-ray image detector 4. Should 3D data sets be produced in accordance with the so-called DynaCT method, the rotatably mounted C-arm 2 with an X-ray emitter 3 and X-ray image detector 4 is rotated in such a way that, as is shown schematically in FIG. 2 overseeing the axis of rotation, the X-ray emitter 3 depicted here as an image through its beam focus, and the X-ray image detector 4, move in an orbit 13 around an object 12 to be examined, which is located in the beam path of the X-ray emitter 3. The orbit 13 can be passed through completely or partially for the creation of a 3D data set.

The C-arm 2 having an X-ray emitter 3 and X-ray image detector 4 thus moves, in accordance with the DynaCT method, preferably around at least one angular region of 180°, for example 180° plus fan angles, and records projection images from various projections in a fast sequence. The reconstruction can only take place from a subdomain of this recorded data.

The examination object 12 can, for example, be an animal or human body, but also a phantom body.

The X-ray emitter 3 and the X-ray image detector 4 each run around the object 5 in such a way that the X-ray emitter 3 and the X-ray image detector 4 face opposite sides of the examination object 12.

In normal radiography or fluoroscopy, the medicinal 2D data of the X-ray image detector 4 is intermediately stored in the imaging system 8, if necessary, by means of such an X-ray diagnostic apparatus, and then reproduced on the monitor 9.

The present problem is the reconstruction of TACs (time attenuation curves), which describe the flow of the contrast medium in the tissue and blood vessels of the brain, from acquisitions with a slowly rotating C-arm angiograph system. The TACs are used to calculate perfusion maps of the brain, such as cerebral blood flow (CBF), cerebral blood volume (CBV) or mean transit time (MTT), which provide important information about the expansion in brain tissue caused by apoplexy. C-arm angiograph systems have a lower rotational speed than computed tomography systems, whereby the temporal resolution of the reconstructed TACs is reduced. Moreover, the contrast attenuation values in the brain tissue are low and are therefore sensitive to noise.

Such a contrast agent course 14 is depicted as an example in FIG. 2. The time in seconds (s) is applied along the X-axis. The Y-axis corresponds to the relative attenuation values in Hounsfield Units (HU). In the example, attenuation values 15 were recorded every two second in sampling points 16. For reasons of clarity, not all attenuation values 15 and not all sampling points 16 were provided with a reference numeral. The attenuation values 15 were recorded as points in the diagram and can serve to calculate the contrast agent course 14 by interpolation. The contrast agent course 14 runs through the measuring points of the attenuation values 15 and produces a constant link between the time and the attenuation at the sampling point.

To determine the contrast agent course 14 of, for example, a test bolus in connection with a pre-examination, a series of samples are fundamentally taken at predetermined sampling points 16 for the determination of the contrast agent course 14, wherein an attenuation value 15 is recorded at each sample, with which a concentration of the contrast agent is represented. The attenuation values 15 of the contrast agent course 14 are stored for the calculation of parameters and forecasting for subsequent examinations of the patient 5.

From the attenuation values 19 of the contrast agent course 17 of, for example, the test bolus, which values have been determined in this way, the necessary operating parameters of the X-ray system can be calculated for the examination. The delay between assigning the contrast agent and starting the recording of filling images is, for example, due to the position of the local maximum 17 of the contrast agent course 14.

In “C-Arm CT Dynamic Cerebral Perfusion Measurement for Ischemic Stroke: An Experimental Study in Canines”, which appeared in Proc. ASNR 50th Annual Meeting, 2012, Royalty et al. shows a dynamic perfusion measurement with C-arm CT in a study that has a fast acquisition protocol with a rotational speed of the C-arm system held by a robot of 1000 U/s.

However, such fast acquisition protocols are not possible in the vast majority of interventional workplaces. Therefore, alternative techniques are required, which enable perfusion measurements from acquisitions with a lower rotational speed.

In “Interventional 4-D C-arm CT Perfusion Imaging Using Interleaved Scanning and Partial Reconstruction Interpolation”, IEEE Trans Med Imaging, 2012, Vol. 31, pages 892 to 906, Fieselmann et al. proposes a new scanning protocol that combines interleaved scanning with partial reconstruction interpolation.

With improved temporal sampling and high computational efficiency, many scanning sequences are required, which enable the irradiation and contrast agent dose of the patient to increase. Also, the adoption of constant hemodynamic behavior between the interleaved acquisitions is, however, not guaranteed in reality.

“Jacobi-like Solution to the Model Based Tomographic X-Ray Perfusion Imaging” by Serowy et al., IEEE Nuclear Science Symposium Conference Record, 2007, and “An iterative method for tomographic x-ray perfusion estimation in a decomposition model-based approach”, by Neukirchen et al., Medical Physics, 2010, Vol. 37, pages 6125 to 6141, are established as iterative model-based approximations that describe reconstructed TAC curves through a sum of weightedly-filtered basis functions in order to keep the degree of freedom relatively low. Although these algorithms can be implemented for similarly classical algebraic reconstruction techniques, the use of basis functions with non-compact support renders additional, expensive, CPU-intensive steps necessary. In each step of forward projection, the dynamic volume must be interpolated as a weighted sum of all weighted base volumes, and each step of the rearward projection must be repeated for all weighted volumes.

In “An extended temporal interpolation approach for dynamic object reconstruction”, Proceedings 11th Fully 3D, 2011, pages 379 to 382, Neukirchen presents a computationally fast, analytical estimation for the interpolation of the projection data that is lacking for an accurate reconstruction. Here, curves of the least squares are adjusted/adapted/brought into conformity with Fourier basis functions between projections, which are scanned or recorded at the same angular positions of the C-arm. However, the accuracy of the interpolation is limited by the low number of sampling points that are acquired during a perfusion scanning sequence with the C-arm.

In the older patent application DE 10 2011 086 771.6, it is described that, to provide an artifact-free tomogram of a body, projection image data generated by means of a radiation-based projection method is used. Firstly, initial voxel data is predetermined to be a plurality of voxels of the body. From this, artificial projection image data is generated based on a projection regulation that reproduces a course of events of the projection method. By comparing the artificial projection image data with the real projection image data, defective projection data is determined. This is imaged on the basis of a rear-projection regulation that is dependent on the projection regulation, such that defective voxel data is generated. Corrected data, and then with the corrected data, corrected voxel data, is generated from the defective voxel data by means of a gradient-based optimization algorithm.

### SUMMARY OF INVENTION

The invention is based on the object of embodying an angiographic examination method of the type cited in the introduction, such that the method for compensating for the slow rotation speed and for reducing the noise level allows for an adequate reconstruction of contrast intensity curves in a reasonable computing time.

The object for an angiographic examination method of the type cited above is solved according to the invention by the features specified in claim 1. Advantageous embodiments are specified in the dependent claims.

The object for an angiographic examination method is solved according to the invention by the projection images acquired by means of the angiography system being subjected to an FDK reconstruction after a pre-processing, the result of which is filtered using a noise reduction method, by a predetermined number of dynamic, iterative reconstruction steps being carried out, by TACs being reconstructed, which are modeled with a weighted sum of linear basis functions.

The noise reduction method can be advantageously based on bilateral filtering by using temporal maximum-intensity projections of TACs as the control image.

It has also been proven to be expedient if the angiographic examination method has, according to the invention, the following steps:

S1) acquiring data, wherein a plurality of projection images are generated from different directions,
S2) subtracting anatomic structures in the projection space,
S3) carrying out a focused FDK reconstruction,
S4) carrying out a bilateral filtering with the control image,
S5) generating a vessel mask,
S6) initializing weight volumes,
S7) querying whether the maximum number of iterations has been reached,
S8) dynamic iterative reconstruction steps,
S9) carrying out a bilateral filtering with the control image and
S10) ending the calculations and reproducing the determined reconstruction results.

Alternatively, the angiographic examination method can, according to the invention, have the following steps:

Sa) acquiring data, wherein a plurality of projection images are generated from different directions,
Sb) pre-processing the data in the projection space,
Sc) carrying out a focused FDK reconstruction,
Sd) generating a vessel mask in the volume and projection space,
Se) initializing weight volumes,
Sf) carrying out a bilateral filtering with the control image,
Sg) querying whether the maximum number of iterations has been reached,
Sh) dynamic iterative reconstruction steps,
Si) carrying out a bilateral filtering with the control image and
Sj) ending the calculations and reproducing the determined reconstruction results.

Advantageously, linear spline basis functions can be used.

It has been proven to be expedient if an optimization strategy with a modified rear-projection step is introduced in the reconstructed volume.

### BRIEF DESCRIPTION OF THE DRAWINGS

The invention is subsequently illustrated in greater detail using the exemplary embodiments depicted in the drawing: The following are shown:

FIG. 1 a known C-arm angiography system having an industrial robot as a carrying device,

FIG. 2 an exemplifying depiction for the illustration of the rotational angiography,

FIG. 3 a time attenuation curve,

FIG. 4 an acquisition protocol for carrying out rotational angiography,

FIG. 5 a flow diagram of the complete algorithm according to the invention, and

FIG. 6 a flow diagram of an alternative to the complete algorithm according to the invention.

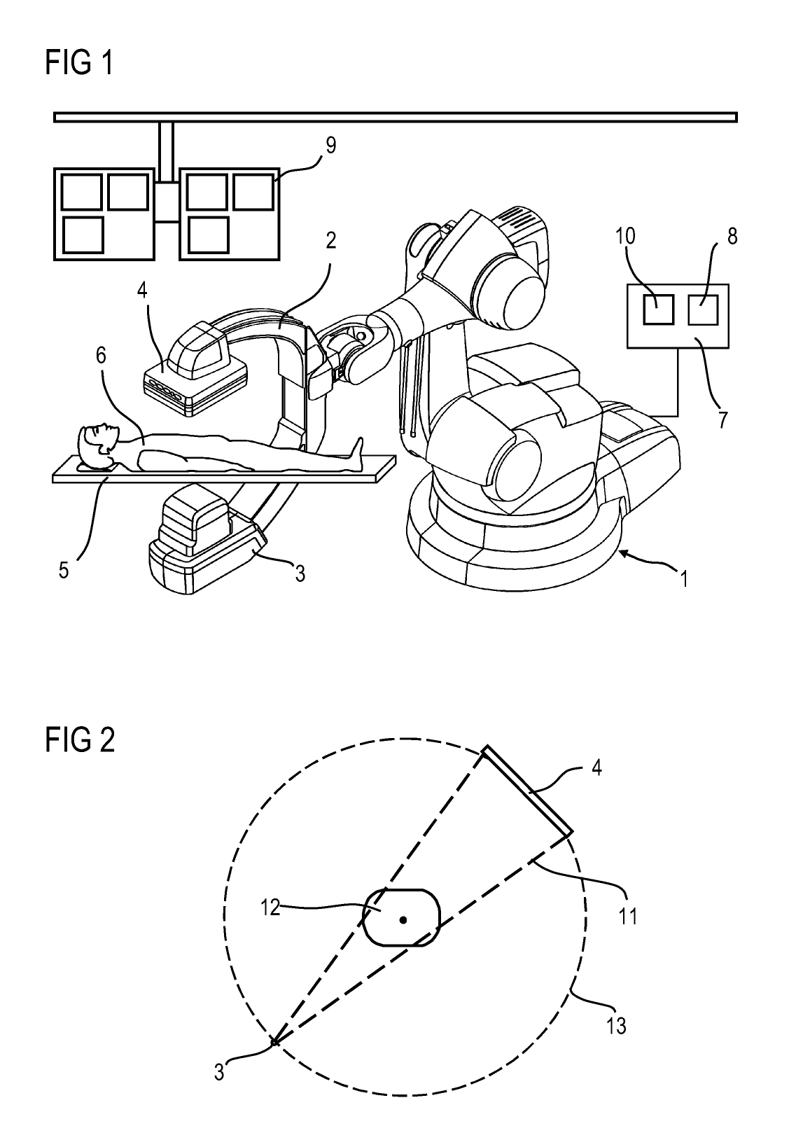

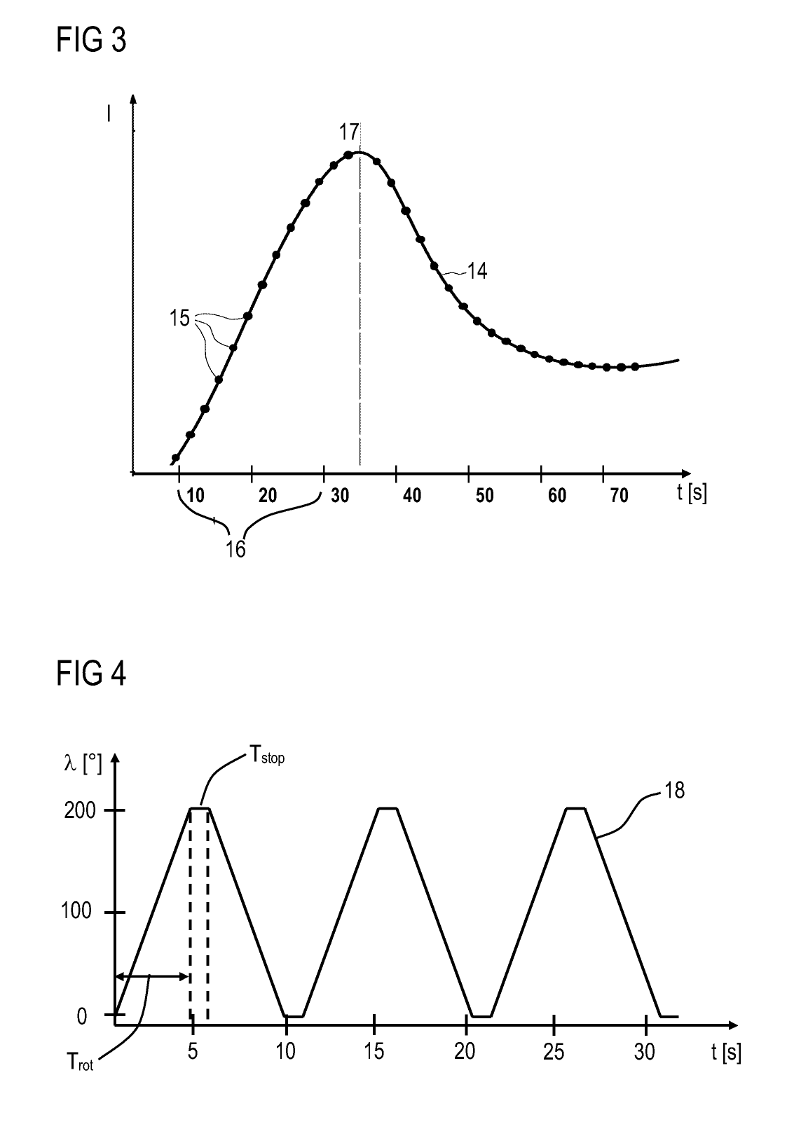

### DETAILED DESCRIPTION OF INVENTION

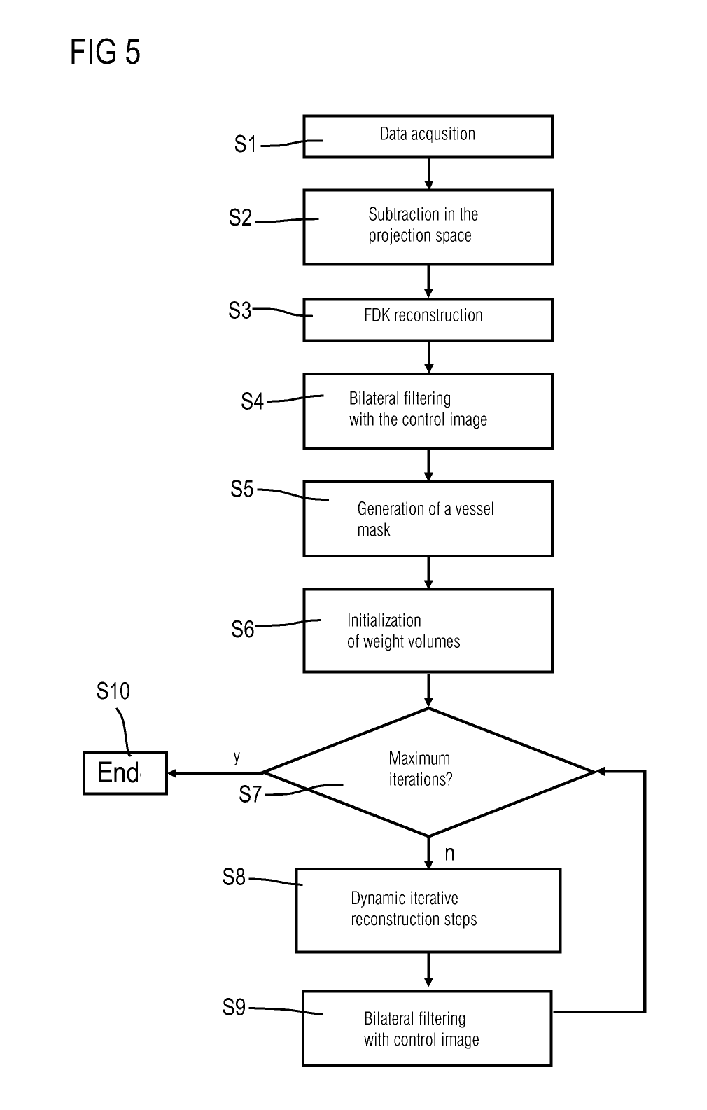

An acquisition protocol 18 for perfusion by means of a C-arm is described in FIG. 4, which can serve for data acquisition. Since the known C-arm systems enable continuous rotation in only one direction, the C-arm is rotated bidirectionally forwards and backwards. The first C-arm rotation in a forward and backward direction acquires basis projections with the static, anatomical structures—a so-called mask. During each rotation, Nₚᵣₒⱼ=248 projections are acquired along an angular region of approx. 200°. After a contrast agent has been injected, the C-arm is rotated approx. Nᵣₒₜ=7 times bidirectionally, as is shown in FIG. 4. Each rotation lasts Tᵣₒₜ=4.3 seconds, with a pause of Tₛₜₒₚ=1.2 seconds between two successive rotations.

Then a direct reconstruction of the rotations would allow a temporal sampling of TACs with a duration of Tₛ=Tᵣₒₜ+Tₛₜₒₚ=5.5 seconds over a total scanning time of T_{scan}=Nᵣₒₜ*Tᵣₒₜ+(Nᵣₒₜ−1)*Tₛₜₒₚ=37.3 seconds. The basis projections are subtracted from the projections of the filling recording after logarithmic pre-processing under the assumption that the examination object 12 has not moved during the acquisition. This generates a projection data vector p=[p₁^{T} . . . p_{N}_{ₚ}^{T}]^{T}, pε [symbol] ^{S}^{ₚ}^{·N}^{ₚ}, which contains only the clear contrast dynamic and the noise, wherein S_{P}=Nᵤ·Nᵥ denotes the size of the detector in pixels with Nᵤ=616 columns and Nᵥ=480 rows after the binning and Nₚ=Nᵣₒₜ·Nₚᵣₒⱼ denotes the total number of acquired contrast-enhanced filling images. Furthermore, the vector tP=[t₁^{p} . . . t_{N}_{ₚ}^{p}] describes the acquisition times of each projection in P.

The acquisition protocol 18 can, for example, have the following acquisition parameters:

Difference angle=0.8°

Number of projections Nₚᵣₒⱼ=248

Angular region per rotationλ=197.6°

Rotation time Tᵣₒₜ=4.3 s

Time between the rotations Tₛₜₒₚ=1.2 s

Number of rotations Nᵣₒₜ=7

Total scanning time T_{scan}=37.3 s

Source-to-Detector Distance (SDD)=1200 mm

Detector pixel size=0.616*0.616 mm²

Number of detector pixels (Nu*Nv)=616*480

Total detector size after 4*4 Rebinning=380*296 mm²

Tube voltage 70 kVp

System dose 1.2 Gy/Projection

Dynamic iterative reconstruction (DIR):

The dynamic iterative reconstruction algorithm is illustrated in greater detail below:

There is a continuous contrast agent flow during the acquisition, such that the observed volume is different at each of the projection images. For an exact resolution, a 4D volume vector x=[x₁^{T} . . . xₙ_{ₚ}^{T}]^{T}, xᵢε [symbol] ^{S}^{ᵥ} must be reconstructed, which consists of Nₚ 3-D volumes Xᵢε [symbol] ^{N}^{ₓ}^{×N}^{_{y}}^{×N}^{_{z}}i=1 . . . Nₚ, described below by a column vector xᵢε [symbol] ^{S}^{ᵥ}, Sᵥ=Nₓ·N_{y}·N_{z}, wherein each voxel in a volume Xᵢ represents a sampling value of a reconstructed TAC. To describe the imaging of the 4D volume onto the projection data, a system matrix A compiled from matrices Aᵢ is defined, which image the 3D volumes onto projection line integrals according to the acquisition geometry p=Ax.

$$A = ( \begin{matrix}{A}_{1} & 0 & … & 0 \\ 0 & {A}_{2} & ⋱ & 0 \\ ⋮ & ⋮ & ⋱ & ⋮ \\ 0 & 0 & … & {A}_{{N}_{p}}\end{matrix} ) \\ with \\ \begin{matrix}{A}_{i} ∈ {{S}_{p} \times {S}_{v}}^{} \\ A ∈ {( {N}_{p} \cdot {S}_{p} ) \times ( {N}_{p} \cdot {S}_{v} )}^{}\end{matrix} \qquad ( 1 )$$

Of course, the direct calculation of the exact resolution x is not possible, since the equation system is heavily under-defined. Therefore, we are limiting the TACs described by x in such a way that they are located within the sub-space that is spanned by linear spline functions, such that

x=Bw with Bε [symbol] ^{(N}^{ₚ}^{·S}^{ᵥ}^{)×(N}^{_{w}}^{·S}^{ᵥ}⁾

wherein w describes the spline weights

w=[w₁^{T} . . . w_{N}_{_{W}}^{T}]^{T},wⱼε [symbol] ^{S}^{_{v.}}

The number of spline basis functions is N_{w}=2·Nᵣₒₜ and the weighting vector wⱼ describes the contrast attenuation at the points in time tⱼ^{w}, wherein

$${t}_{j}^{w} \{ \begin{matrix}⌊ \frac{j - 1}{2} ⌋ \cdot ( {T}_{stop} + {T}_{rot} ) + 0 , 25 \cdot {T}_{rot} & j = gerade \\ ⌊ \frac{j - 1}{2} ⌋ \cdot ( {T}_{stop} + {T}_{rot} ) + 0 , 75 \cdot {T}_{rot} & j = ungerade\end{matrix} applies .$$

So, the point in time tⱼ^{w} describes the temporal position of the nodes of the linear spline that belong to the weighting vector wⱼ. The base matrix B calculates the volume vector xᵢ by linear interpolation between the two closest weighting vectors.

$${x}_{i} = ( 1 - {w}_{i} ) {w}_{p} + {w}_{i} {w}_{n} \\ with \\ {w}_{i} = \frac{{t}_{i}^{P} - {t}_{p}^{w}}{{t}_{n}^{w} - {t}_{p}^{w}} \\ and \\ \begin{matrix}p = max \{ p \vert {t}_{p}^{w} < {t}_{i}^{p} , p = 1 … {N}_{w} \} \\ n = min \{ n \vert {t}_{n}^{w} \geq {t}_{i}^{P} , n = 1 … {N}_{w} \}\end{matrix} \qquad ( 2 )$$

Exceptions must be defined for the beginning and end of an acquisition:

$$If$$

$${t}_{i}^{P} \leq {t}_{1}^{w}$$

$$then$$

$${x}_{i} = \frac{{t}_{i}^{P}}{{t}_{1}^{w}} {w}_{1}$$

$$and$$

$$If$$

$${t}_{i}^{P} > {t}_{{N}_{w}}^{w}$$

$$then$$

$${x}_{i} = {w}_{{N}_{w}} .$$

This reflects the assumption that we have an increase in contrast attenuation from 0 HU at the start and a constant flat phase of the remaining contrast at the end.

The problem of optimization is given by the problem of the smallest square:

$$\hat{w} = {arg min}_{w} {‖ ABw - p ‖}_{2} \qquad ( 3 )$$

As described by Neukirchen et al., we solve this significant problem by using a gradient-based iterative procedure, based on the Landweber scheme. This results in a weighted update step, similar to the classical ART-based algorithm for static data:

w^{k+1}=wk+β·B^{T}A^{T}(ABw^{k}−p) (4)

The relaxation parameter β controls the increment of the parameter updates in each iteration. AB describes a linear interpolation, followed by a forward projection and BA is a weighted rearward projection of the defect pattern onto the base weightings.

The calculation of the spline weights w is carried out in a similar way as is described in Neukirchen et al. The 3D weighting volumes Wⱼε [symbol] ^{N}^{ₓ}^{×N}^{_{y}}^{×N}^{_{z}} are reconstructed, wherein each weight volume Wⱼ represents the weightings in the vector wⱼ as 3D volumes, by using a beam-operated forward projection and a voxel-operated rearward projection. An ordered subset estimation is used to improve the convergence speed. The projection of each rotation is divided into ten different subsets, which maximize the difference in acquisition angle in each subset. In each interaction, the algorithm processes the projections of all rotations successively. For each projection p, the corresponding volume Xᵢ is calculated according to the equation by using a GPU-implemented linear interpolation, projected forwards and subtracted by the measured projection images. The resulting defective image is, weighted with its corresponding basis function values (1-w) and w, projected onto the corresponding weighted volumes Wₚ and Wₙ. After a subset of projections has been processed, all negative attenuation weights in the updated vector are set to zero in order to ensure a physically correct resolution.

The direct application of the optimization strategy by Neukirchen et al., in combination with linear basis functions, converges slowly and the reconstructions are distorted by stripe artifacts. The maximum intensity projection (MIP) is generated by the use of the maxima of the reconstructed TACs. Therefore, a good initialization and a sensible optimization strategy are necessary, which ensure that the algorithm converges on a useful result. For this, all rotations are first reconstructed with the FDK algorithm. A sharp filter kernel (σ_{K}=0.25) is used to prevent blurring of the high-contrast vessels in the soft tissue. From the FDK reconstruction, initial TACs are calculated by linear interpolation, wherein each reconstructed rotation represents examples of TACs at a temporally central point in time of its acquisition. The weight volumes Wⱼ are initialized by using the interpolated TACs. To avoid stripe artifacts, the rear-projection step is modified. For this, the temporal MIP is calculated from the initial TACs. A vessel mask is generated in the volume space V^{v}(v): N³→{0,1} by forming threshold values of the maximum intensity projection (MIP) with the threshold τ_{MIP}, which shows which voxels belong to which vessel. Accordingly, the vessel masks in the projection space Vᵢ^{v}(u): N²→{0,1} for all i=1 . . . Nₚ projections are calculated by a maximum intensity forward projection of V^{v}. The projection vessel mask displays which detector pixels u belong to a beam, which pixels are cut with a vessel structure. In all rear projection steps, pixels in the defective image, in connection with a vessel-cutting beam, are rear-projected through Vᵢ^{P} only onto voxels that belong to a vessel according to V^{v}. This helps to avoid several stripe artifacts that usually arise in a resulting MIP screen from the vessel-masked reconstruction.

The rear projection is therefore modified to prevent stripe artifacts in the vicinity of high-contrast vessels. To that end, a vessel mask in the volume space and vessel masks in the projection space are produced. During the rear projection, beams that pass through a vessel (displayed by the vessel masks in the projection space) are only projected onto vessel voxels (displayed by vessel masks in the volume space).

Bilateral filtering with the control image (JBF—joint bilateral filtering):

Due to the high noise sensitivity of the perfusion images, a sensible regularization for the algorithm is necessary, which enables a robust reconstruction of the TACs under noisy conditions. To that end, a bilateral filtering with the control image is used (joint bilateral filtering)—a non-linear, edged denoising filter that uses a combination of location and intensity filtering. To determine the similarity in intensity of voxels, the maxima of the TACs are used, so the temporal maximum intensity projection (MIP). This results in a bilateral filtering, wherein the similarity in intensity is calculated by the temporal maximum intensity projection instead of the filtered volume itself. Such a filtering, wherein a different image is used to calculate the similarity in intensity, is denoted as a joint bilateral filter. The filtered weight volume Wⱼ^{JBF} is calculated from the original volume Wⱼ by

$${W}_{j}^{JBF} ( v ) = {k}^{- 1} ( v ) {\sum }_{{v}^{′} ∈ {N}_{v}}^{ } {W}_{j} ( v ) c ( v , {v}^{′} ) s ( M ( v ) , M ( {v}^{′} ) ) , \\ s ( M ( v ) , M ( {v}^{′} ) ) = exp ( - {‖ M ( v ) - M ( {v}^{′} ) ‖}_{2}^{2} / {σ}_{R}^{2} ) , \\ c ( v , {v}^{′} ) = exp ( - {‖ v - {v}^{′} ‖}_{2}^{2} / {σ}_{D}^{2} ) , \\ k ( v ) = {\sum }_{{v}^{′} ∈ {N}_{v}}^{ } c ( v , {v}^{′} ) s ( M ( v ) , M ( {v}^{′} ) ) . \qquad ( 5 )$$

Each voxel v of the filtered volume Wⱼ^{JBF} is a combination of voxels of the original volume Wⱼ, which belong to the vicinity Nᵥ, weighted with the MIP similarity s and the spatial proximity c and normalized by division with the sum of all weights k.

In FIG. 5, the procedure of the method according to the invention is depicted in greater detail in the form of a flow diagram.

An acquisition of data, for example by means of a C-arm angiography system, is carried out as the first method step S1), wherein a plurality of projection images are generated from different directions.

The anatomical structures in the projection space are subtracted in a second method step S2).

Then, in a third method step S3), a so-called sharp FDK reconstruction is carried out—a Feldcamp reconstruction with a so-called sharp filter kernel (σ_{K}=0.25).

These volume images obtained in such a way are subjected, in a fourth method step S4), to bilateral filtering with the control image.

Then, in a fifth method step S5), vessel masks—masking images of the vessels—are generated in the volume space and in the projection space.

The weight volumes are initialized in a sixth method step S6).

In a query according to a seventh method step S7), it is determined whether the maximum desired number of iterations has been reached.

If this is not the case, dynamic iterative reconstruction steps are applied to the volumes in the eighth method step S8).

In a final calculation step S9), the reconstruction results are subjected to a bilateral filtering with the control image.

At the end, if the response in method step S7) is positive, the determined reconstructions are reproduced in a tenth method step S10).

Firstly, the basis projections with the static anatomical structures are subtracted from the contrast-enhanced projections—the filling images. Then all rotations with the FDK algorithm are reconstructed with a sharp filter kernel. In a next step, an initial MIP volume M is calculated. Then the noise in all initial volumes is reduced by means of the bilateral filtering with the control image and an updated volume M is determined from these noise-reduced reconstructions. The vessel masks for all projections of the forward and rearward projections are calculated by forward projections of the volume vessel masks by using a maximum intensity forward projection. After the initialization of the weight volumes from the FDK reconstructions that are liberated from noise, a fixed number of iterations are carried out. Each iteration consists of a DIR step to achieve data consistency between the weight volumes and the measured projection data, following the denoising of all weight volumes with bilateral filtering with the control image.

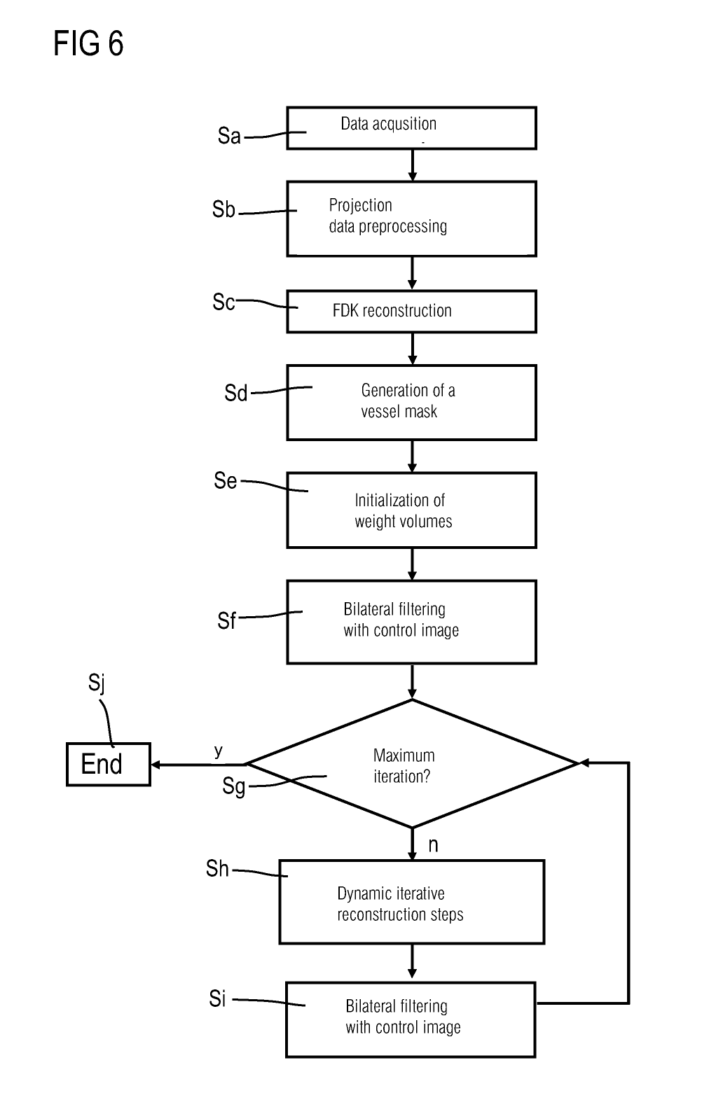

FIG. 6 shows a procedure of an alternative to the method according to the invention in the form of a flow diagram.

An acquisition of data, for example by means of the C-arm angiography system, is carried out as a first method step Sa), wherein several projection images are generated from different directions.

In a second method step Sb), a pre-processing of the data takes place in the projection space.

Then, in a third method step Sc), a so-called sharp FDK reconstruction is carried out—a Feldcamp reconstruction with a so-called sharp filter kernel (σ_{K}=0.25).

Then, in a fourth method step Sd), vessel masks—masking images of the vessels—are generated in the volume space and in the projection space.

In a fifth method step Se), an initialization of weight volumes is carried out.

These volume images obtained in such a way are subjected, in a sixth method step Sf), to bilateral filtering with the control image.

In a query according to a seventh method step Sg), it is determined whether the maximum desired number of iterations has been reached.

If this is not the case, dynamic iterative reconstruction steps are applied to the volumes in the eighth method step Sh).

In a final calculation step Si), the reconstruction results are subjected to a bilateral filtering with the control image.

At the end, in a tenth method step Sj), the determined reconstructions are reproduced.

In this alternative to the complete algorithm, projection images are thus first generated. After the pre-processing of the data in the projection space, all rotations are reconstructed by means of the FDK algorithm. In a next step, a volume mask is calculated in the volume and projection space and the weight volume is initialized. Then, all weight volumes are denoised by means of JB filtering. A fixed number of iterations are carried out hereafter. Each iteration consists of a DIR step in order to ensure the data consistency between the weight volumes and the measured projection data, followed by denoising of all weight volumes with filtering. The algorithm parameters for the majority of the experiments are specified below.

For the reconstruction of time attenuation curves with an improved temporal resolution, the TACs are modeled by a weighted sum of linear spline functions and the algorithm calculates the basis weighting from the acquired data. During the reconstruction, a denoising strategy based on bilateral filtering with the control image is applied. The main step of the algorithm has been illustrated in greater detail by FIGS. 5 and 6.

A dynamic iterative algorithm is proposed, which reconstructs TACs and which is modeled with a weighted sum of linear spline basis functions. Compared to known estimations, the use of linear spline basis functions reduces the calculation time, which is an important factor in interventional imaging.

To avoid vessel structures with high-contrast-surrounded stripe artifacts in the reconstructed volume, a new optimization strategy with a modified rear-projection step is introduced.

A new noise-reducing method, based on bilateral filtering with the control image by using the temporal maximum intensity projection of the TACs as the control image, is described. The bilateral filtering with the control image provides mathematically fast, stable and advantageous regulation that is also easy to implement.

To compensate for the slow rotation speed, and to reduce the noise level, a software algorithm is described, which enables an adequate reconstruction of TACs in a measured calculation time.

1. An angiographic examination method for examining a region of a patient body by an angiography system, wherein the angiography system comprises an X-ray tube assembly and an X-ray image detector being applied to ends of a C-arm, a patient table having a tabletop for carrying the patient, a system control unit, an imaging system and a monitor, the method comprising:
generating projection images by the angiography system via rotational angiography from a plurality of projection angles in a projection space;
reconstructing the projection images by an FDK reconstruction after a pre-processing to generate a 3D data set in a volume space; and
filtering the reconstructed projection images using a noise reduction method,
wherein a predetermined number of dynamic iterative reconstruction steps are carried out, and
wherein time attenuation curves are reconstructed that are modeled with a weighted sum of linear basis functions.

2. The angiographic examination method as claimed in claim 1, wherein the noise reduction method is based on bilateral filtering by using temporal maximum intensity projections of the time attenuation curves as a control image.

3. The angiographic examination method as claimed in claim 1, further comprising:
subtracting anatomic structures in the projection images in the projection space,
reconstructing the projection images by a sharp FDK reconstruction,
bilateral filtering the reconstructed projection images with a control image,
generating a vessel mask,
initializing weight volumes,
querying whether the predetermined number of dynamic iterative reconstruction steps has been reached,
dynamically iterative the reconstructing step,
bilateral filtering the reconstructed projection images with the control image, and
ending the iteration and reproducing the reconstructed projection images.

4. The angiographic examination method as claimed in claim 1, further comprising:
pre-processing the projection images in the projection space,
reconstructing the projection images by a sharp FDK reconstruction,
generating a vessel mask in the volume space and the projection space,
initializing weight volumes,
bilateral filtering the reconstructed projection images with a control image,
querying whether the predetermined number of dynamic iterative reconstruction steps has been reached,
dynamically iterative the reconstructing step,
bilateral filtering the reconstructed projection images with the control image, and
ending the iteration and reproducing the reconstructed projection images.

5. The angiographic examination method as claimed in claim 1, wherein the linear basis functions comprise linear spline basis functions.

6. The angiographic examination method as claimed in claim 1, wherein the reconstructing comprises an optimization strategy with a modified back projection step.
